(19) United States
(12) Patent Application Publication
(10) Pub. No.: US 2026/0171191 A1
(43) Pub. Date: Jun. 18, 2026

(54) APPARATUS AND METHOD FOR ANALYZING CELLS BY USING STATE INFORMATION OF CHROMOSOME STRUCTURE

(71) Applicants: CLINOMICS INC., Ulsan (KR); UNIST (ULSAN NATIONAL INSTITUTE OF SCIENCE AND TECHNOLOGY), Ulsan (KR)

(72) Inventors: Byoung Chul Kim, Ulsan (KR); Jong Hwa Bhak, Ulsan (KR); Chang Jae Kim, Ulsan (KR); Ji Hye Ahn, Ulsan (KR); Hyo Jin Um, Ulsan (KR); Ha Hyeon Jeon, Ulsan (KR); Yeo Jin Kim, Gyeongsangnam-do (KR)

(21) Appl. No.: 18/710,502

(22) PCT Filed: Nov. 3, 2022
(86) PCT No.: PCT/KR2022/017157

§ 371 Date: May 15, 2024

(30) Foreign Application Priority Data
Nov. 19, 2021 (KR) 10-2021-0160058

(51) Int. Cl. G16B 30/10; G16B 15/00; G16B 40/00

(52) U.S. Cl. CPC G16B 30/10; G16B 15/00; G16B 40/00

(57) ABSTRACT

Disclosed are an apparatus and method for analyzing cells by using state information of a chromosome structure. The present invention determines whether diseased cells are present in a cell group collected from a sample of a subject through state analysis of a chromosome structure, and predicts the tissue origin and quantity of the diseased cells. According to the present invention, it is possible to classify diseased cells with high accuracy at a low price, and to perform quantitative measurement more easily and accurately than conventional cell staining methods.

### TECHNICAL FIELD

The present invention relates to an apparatus and method for analyzing cells for predicting and diagnosing diseases, etc. by finding and comparing information on state modifications or changes such as opening, closing, and the like on a chromosome structure, and more particularly, to determine whether diseased cells are present in a cell group collected from a sample of a subject, and analyze modifications and changes according to the degree of opening and closing of the chromosome structure. Further, the present invention relates to an apparatus and method for predicting the tissue origin and quantity of the diseased cells.

### BACKGROUND ART

In the related art, circulating tumor cells (CTCs) in the blood, epithelial cells of organs, or the like are identified using simple and specialized biomarkers. However, the circulating tumor cells (CTCs) in the blood, the epithelial cells of organs, or the like are present in very small amounts in the blood and urine of cancer patients or patients with inflammation or heart disease, and thus even if the cells are enriched using a liquid biopsy analysis device or kit, there is a problem that makes accurate detection difficult.

### DISCLOSURE

### Technical Problem

An object of the present invention is to provide an apparatus and method for analyzing cells by using information of a chromosome structure by analyzing a state of a chromosome structure and patterns of the state to determine whether diseased cells are present in a cell group collected from a sample of a subject, and predict the tissue origin and quantity of the diseased cells.

### Technical Solution

An aspect of the present invention provides a method for analyzing cells by using state information of a chromosome structure, the method including: obtaining a state of a genome structure of cells collected from a sample; analyzing a state modification region of the genome structure based on a pre-stored standard genome structure state pattern DB, and classifying the collected cells into diseased cells and normal cells; analyzing a modification or change region of opening, closing, and the like of the genome structure state based on a pre-stored genome structure state pattern DB for each tissue, and obtaining the tissue origin of the diseased cells; and analyzing a modification or change region of the genome structure state based on the standard genome structure state pattern DB and the genome structure state pattern DB for each tissue, and obtaining the quantity of the diseased cells. The modification refers to a modification in the storage state of a chromosome compared to a normal and the like, and as a result, a relatively occurring change is called a state change of the structure on the chromosome.

The classifying may consist of classifying the collected cells into the diseased cells and the normal cells by comparing the state of the genome structure stored in the standard genome structure state pattern DB with the state of the genome structure of the collected cells, based on the number of sequences and peaks of the state modification region of the genome structure.

The obtaining of the tissue origin may consist of obtaining the tissue origin of the diseased cells by comparing the state of the genome structure stored in the genome structure state pattern DB for each tissue with the state of the genome structure of the collected cells, based on a peak pattern of the state modification region of the genome structure.

The obtaining of the tissue origin may consist of obtaining the tissue origin of the diseased cells by comparing a peak position of the genome structure stored in the genome structure state pattern DB for each tissue with a peak position of the genome structure of the collected cells.

The obtaining of the tissue origin may consist of obtaining the tissue origin of the diseased cells based on a ratio in which a peak region of the genome structure stored in the genome structure state pattern DB for each tissue overlaps with a peak region of the genome structure of the collected cells.

The obtaining of the tissue origin may consist of obtaining the tissue origin of the diseased cells by comparing a matrix obtained based on a peak score of the genome structure stored in the genome structure state pattern DB for each tissue with a matrix obtained based on a peak score of the genome structure of the collected cells.

The obtaining of the quantity of the diseased cells may consist of obtaining the quantity of the diseased cells by calculating the number of diseased cells compared to the total number of cells, by using the number of sequences obtained for a state modification region of a specific genomic structure of the diseased cells and the number of sequences obtained for a state modification region of a specific genomic structure of the normal cells, based on the standard genome structure state pattern DB and the genome structure state pattern DB for each tissue.

Another aspect of the present invention provides an apparatus for analyzing cells by using state or state change information of a chromosome structure, the apparatus including: a cell analysis unit configured to obtain a state of a genome structure of cells collected from a sample; a cell classification unit configured to analyze a state modification region of the genome structure based on a pre-stored standard genome structure state pattern DB, and classify the collected cells into diseased cells and normal cells; a cell origin obtainment unit configured to analyze a state modification region of the genome structure based on a pre-stored genome structure state pattern DB for each tissue, and obtain the tissue origin of the diseased cells; and a cell quantity obtainment unit configured to analyze a state modification region of the genome structure based on the standard genome structure state pattern DB and the genome structure state pattern DB for each tissue, and obtain the quantity of the diseased cells.

The cell classifying unit may classify the collected cells into the diseased cells and the normal cells by comparing the state of the genome structure stored in the standard genome structure state pattern DB with the state of the genome structure of the collected cells, based on the number of sequences and peaks of the state modification region of the genome structure.

The cell origin obtainment unit may obtain the tissue origin of the diseased cells by comparing the state of the genome structure stored in the genome structure state pattern DB for each tissue with the state of the genome structure of the collected cells, based on a peak pattern of the state modification region of the genome structure.

The cell origin obtainment unit may obtain the tissue origin of the diseased cells by comparing a peak position of the genome structure stored in the genome structure state pattern DB for each tissue with a peak position of the genome structure of the collected cells.

The cell origin obtainment unit may obtain the tissue origin of the diseased cells based on a ratio in which a peak region of the genome structure stored in the genome structure state pattern DB for each tissue overlaps with a peak region of the genome structure of the collected cells.

The cell origin obtainment unit may obtain the tissue origin of the diseased cells by comparing a matrix obtained based on a peak score of the genome structure stored in the genome structure state pattern DB for each tissue with a matrix obtained based on a peak score of the genome structure of the collected cells.

The cell quantity obtainment unit may obtain the quantity of the diseased cells by calculating the number of diseased cells compared to the total number of cells, by using the number of sequences obtained for a state modification region of a specific genomic structure of the diseased cells and the number of sequences obtained for a state modification region of a specific genomic structure of the normal cells, based on the standard genome structure state pattern DB and the genome structure state pattern DB for each tissue.

### Advantageous Effects

According to the apparatus and method for analyzing the cells by using the state information of the chromosome structure according to the present invention, it is possible to classify diseased cells with high accuracy at a low price by determining whether diseased cells are present in a cell group collected from a sample of a subject through state analysis of the chromosome structure.

In addition, it is possible to perform quantitative measurement more easily and accurately than conventional cell staining methods by predicting the tissue origin and quantity of the diseased cells.

Further, among diseased cells, the state modification region of the genome structure of circulating tumor cells (CTC) is a region highly associated with genetic and epigenetic modifications in disease-derived cells such as cancer cells, and the state modification region is analyzed to be associated and applied to various other cancer molecule markers, and this method may be applied to other diseases such as heart disease using the same principle.

In addition, through genome structure-based analysis of the diseased cells, it is possible to be easily linked to the development of multi-omics multiple markers, such as structure-associated disease gene function markers, epigenomic markers, and mutation markers.

### DESCRIPTION OF DRAWINGS

FIG. 1 is a block diagram illustrating an apparatus for analyzing cells by using state information of a chromosome structure according to a preferred embodiment of the present invention.

FIG. 2 is a diagram for describing the state information of the chromosome structure.

FIG. 3 is a diagram for describing a difference in state information of the chromosome structure for each cell line according to a tissue type.

FIG. 4 is a diagram for describing an example of decoding a euchromatin region using ATAC-seq according to the present invention.

FIG. 5 is a diagram for describing the number of sequences and peaks of a state modification region of a genome structure according to the present invention.

FIG. 6 is a diagram for describing a comparative example of a standard white blood cell genome structure and a genome structure of diseased cells according to the present invention.

FIG. 7 is a diagram for describing comparative examples of patterns of tissue/disease-specific genome structures according to the present invention.

FIG. 8 is a diagram for describing the comparison of genome structure patterns using peak positions of the genome structure according to the present invention.

FIG. 9 is a diagram for describing the comparison of genome structure patterns using a peak region overlap ratio of the genome structure according to an exemplary embodiment of the present invention.

FIG. 10 is a diagram for describing the comparison of genome structure patterns using peak scores of the genome structure according to the present invention.

FIG. 11 is a flowchart for describing a method for analyzing cells by using state information of a chromosome structure according to a preferred embodiment of the present invention.

FIG. 12 is a flowchart illustrating exemplary experimental steps for confirming through experimental data that collected cells may be classified into normal cells and diseased cells by analyzing a state modification region of a genome structure of the collected cells according to the present invention.

FIG. 13 illustrates a result of analyzing sequencing data for cells isolated from a diseased cell sample, and FIG. 14 illustrates a result of analyzing sequencing data for cells isolated from a normal cell sample.

FIG. 15 illustrates a result of analyzing sequencing data for cells isolated from Experimental group sample 1, and FIG. 16 illustrates a result of analyzing sequencing data for cells isolated from Experimental group sample 3.

FIG. 17 is a graph showing comparing the sizes of peaks detected in sequencing data of cells isolated from a normal cell sample and Experimental group sample 3 at positions of the same chromosome.

FIG. 18 illustrates result data of analyzing diseased cell-specific regions which are not shown in a normal cell sample in sequences obtained by sequencing Experimental group sample 1 using a computer program, FIG. 19 illustrates result data of analyzing diseased cell-specific regions which are not shown in a normal cell sample in sequences obtained by sequencing Experimental group sample 2 using a computer program, and FIG. 20 illustrates result data of analyzing diseased cell-specific regions which are not shown in a normal cell sample in sequences obtained by sequencing Experimental group sample 3 using a computer program.

### MODES OF THE INVENTION

Hereinafter, preferred embodiments of an apparatus and method for analyzing cells by using state information of a chromosome structure according to the present invention will be described in detail with reference to the accompanying drawings.

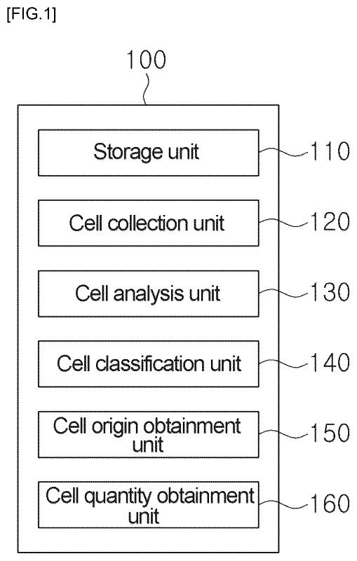

First, an apparatus for analyzing cells by using state information of a chromosome structure according to a preferred embodiment of the present invention will be described with reference to FIGS. 1 to 3.

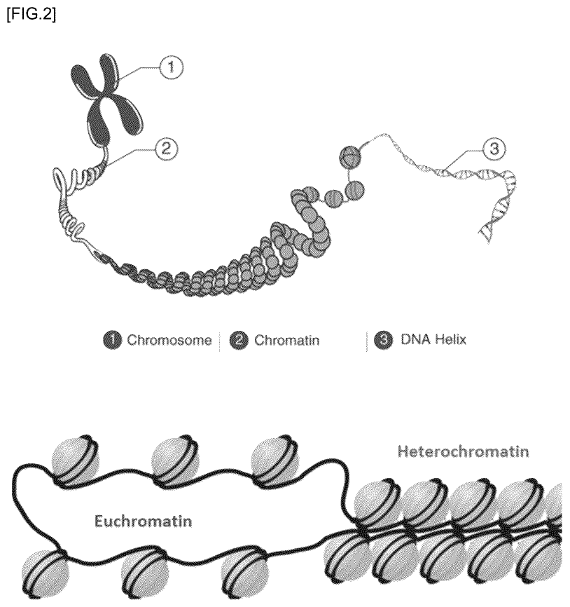

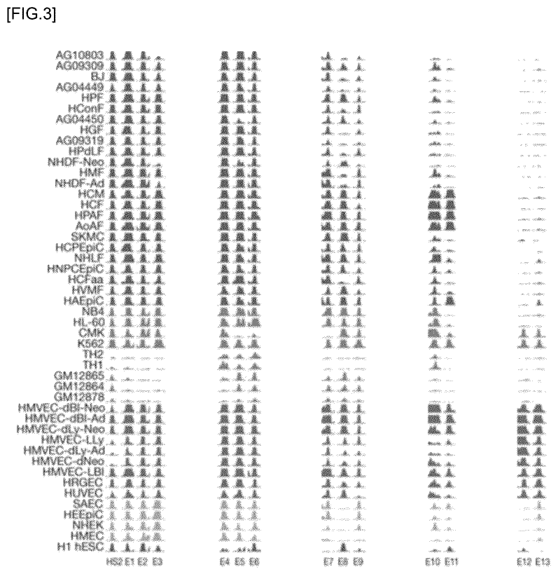

FIG. 1 is a block diagram illustrating an apparatus for analyzing cells by using state information of a chromosome structure according to a preferred embodiment of the present invention, FIG. 2 is a diagram for describing the state information of the chromosome structure, and FIG. 3 is a diagram for describing a difference in state information of the chromosome structure for each cell line according to a tissue type.

Referring to FIG. 1, an apparatus for analyzing cells by using state information of a chromosome structure according to a preferred embodiment of the present invention (hereinafter, referred to as a ‘cell analysis apparatus’) 100 determines whether diseased cells (e.g., circulating tumor cells, circulating cardiovascular adventitial cells, circulating inflammatory disease epithelial cells, etc.) are present in a cell group collected from a sample of a subject and predicts the tissue origin and quantity of the diseased cells.

Here, the state information of the chromosome structure (i.e., genome structure) is commonly called various types of information, such as a sequence of the genome, functional opening of a genome region (open chromatin, euchromatin), comprehensive arrangement, types, and patterns of epigenes, etc. as illustrated in FIG. 2. The euchromatin (open chromatin) is a region where many genes to be expressed are distributed and the chromatin density is relatively low to have transcriptional activity. Heterochromatin (closed chromatin) is a region where gene expression is suppressed due to relatively high chromatin density and low transcriptional activity. Assay for Transposase-Accessible Chromatin using sequencing (ATAC-seq) is used as a method to determine the structural information of these chromosomes. ATAC-seq is a sequence decoding method that identifies a difference in the open/closed states of the chromosome structure, and may profile all euchromatin regions on the genome by decoding regions where chromatin is functionally open.

In addition, as illustrated in FIG. 3, the patterns of euchromatin are characterized by varying depending on the tissue origin of each cell. Accordingly, through analysis of ATAC-seq data, it is possible to profile euchromatin patterns and predict and analyze the tissue origin of specific cells. In other words, ATAC-seq is analyzed in disease-derived cells (circulating tumor cells, circulating inflammatory disease epithelial cells, circulating cardiovascular adventitia cells, etc.) included in the blood of patients with a specific disease, and disease-derived cells may be predicted/analyzed after reading a disease-derived cell-specific genome structure modification region by comparing a genome structure DB of normal white blood cells. For example, circulating tumor cells (CTC) included in the blood of cancer patients may be detected and the tissue origin of the circulating tumor cells may be found through analysis of the state of chromosome structure information.

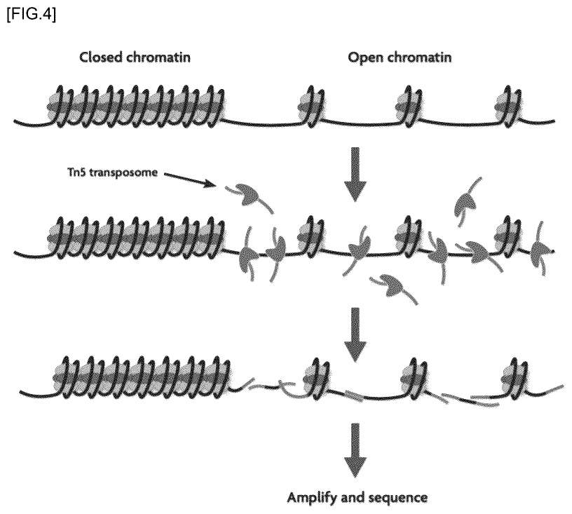

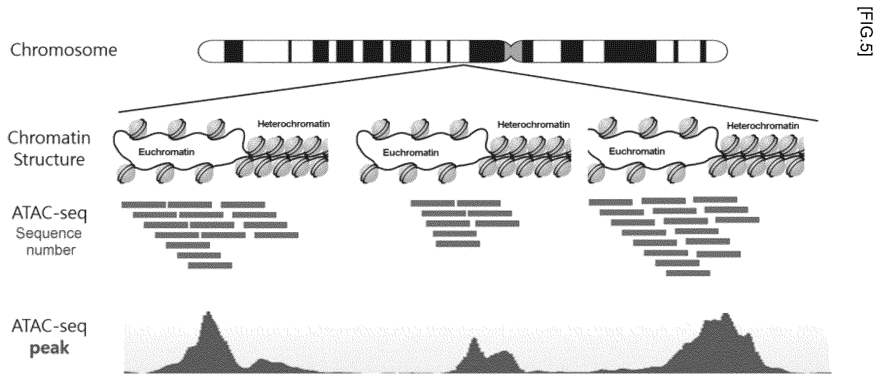

Then, the apparatus for analyzing the cells using the state information of the chromosome structure according to a preferred embodiment of the present invention will be described in more detail with reference to FIGS. 4 to 10.

As illustrated in FIG. 1, the cell analysis apparatus 100 may include a storage unit 110, a cell collection unit 120, a cell analysis unit 130, a cell classification unit 140, a cell origin obtainment unit 150, and a cell quantity obtainment unit 160.

The storage unit 110 stores a standard genome structure pattern database (DB), a genome structure pattern database (DB) for each tissue, etc.

Here, the standard genome structure pattern DB stores state information of the genome structure of white blood cells that may be regarded as normal cells. Since the genome structure pattern of white blood cells may vary for each race, a standard genome structure pattern DB may also be constructed for each race.

In addition, the genome structure pattern DB for each tissue stores state information of the genome structure corresponding to the tissue/disease for each tissue or each disease (e.g., each cancer type, etc.).

At this time, the genome structure stored in the standard genome structure state pattern DB or the genome structure state pattern DB for each tissue may include a euchromatin region structure of the genome, a heterochromatin region structure of the genome, a chromatin cross-link region structure of the genome, a protein-binding region structure of the genome, an epigenomic region structure of the genome, a partial copy number modification region of the genome, etc. For convenience of description, the present invention will be described below assuming that the genome structure is the euchromatin region structure of the genome.

The cell collection unit 120 collects cells from a sample (blood, urine, etc.) of a subject through a liquid biopsy device or kit.

The cell analysis unit 130 obtains state information of the genome structure of cells collected from the sample.

That is, the cell analysis unit 130 may confirm the sequence patterns, structures, and the like on the genome through genome decoding or genotyping of the collected cells.

FIG. 4 is a diagram for describing an example of decoding a euchromatin region using ATAC-seq according to the present invention.

As illustrated in FIG. 4, the cell analysis unit 130 may decode the genome of the collected cells through an ATAC-seq experiment and confirm a euchromatin (open chromatin) region on the genome.

The cell classification unit 140 analyzes a state modification region of the genome structure based on a standard genome structure state pattern DB pre-stored in the storage unit 110, and classifies the collected cells into diseased cells and normal cells.

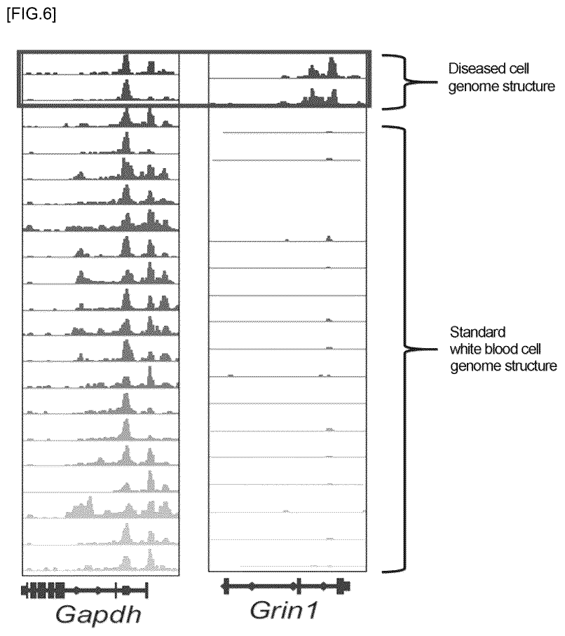

FIG. 5 is a diagram for describing the number of sequences and peaks of a state modification region of a genome structure according to the present invention and FIG. 6 is a diagram for describing a comparative example of a standard white blood cell genome structure and a genome structure of diseased cells according to the present invention.

That is, the cell classification unit 140 may classify the collected cells into diseased cells and normal cells by comparing the genome structure stored in the standard genome structure state pattern DB with the genome structure of the collected cells, based on the number of sequences and peaks of the state modification region of the genome structure as illustrated in FIG. 5.

For example, the cell classification unit 140 may obtain a candidate region to be predicted as a genome structure state region of the diseased cells by analyzing the state modification region of a specific genome structure of the collected cells, that is, comparing the genome structure stored in the standard genome structure state pattern DB with the genome structure of the collected cells to exclude a genome structure state region shown generally in white blood cells. Referring to FIG. 6, it may be seen that there is no difference in genome structure of a Gapdh gene between diseased cells and normal cells (white blood cells), but there is a difference in genome structure of a Grin1 gene between the diseased cells and the normal cells (white blood cells).

The cell origin obtainment unit 150 obtains the tissue origin of the diseased cells classified into the diseased cells through the cell classification unit 140 by analyzing the genome structure modification region, based on the genome structure state pattern DB for each tissue pre-stored in the storage unit 110.

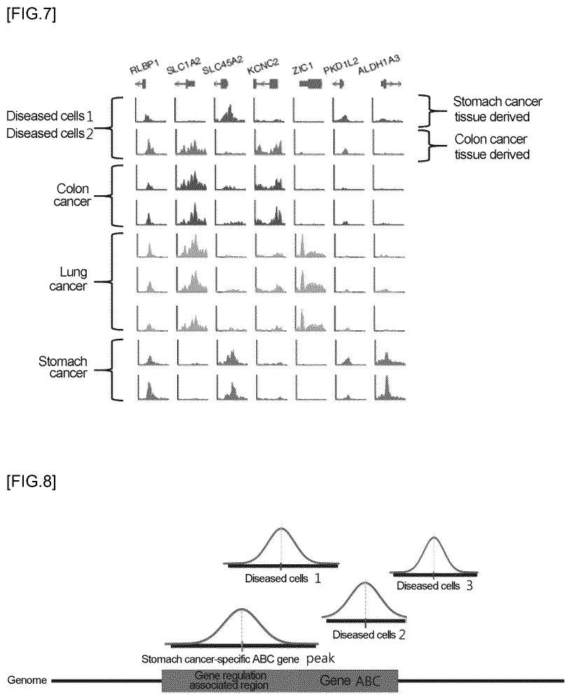

FIG. 7 is a diagram for describing comparative examples of state patterns of tissue/disease-specific genome structures according to the present invention.

That is, the cell origin obtainment unit 150 may obtain the tissue origin of the diseased cells by comparing the genome structure stored in the genome structure state pattern DB for each tissue with the state of the genome structure of the collected cells, based on a peak pattern of the state modification region of the genome structure.

Referring to FIG. 7, it may be seen that a peak pattern of diseased cells 1 is similar to a peak pattern of a state modification region of a genome structure of stomach cancer, so that the tissue origin of diseased cells 1 is stomach cancer (i.e., stomach), and it may be seen that a peak pattern of diseased cells 2 is similar to a peak pattern of a state modification region of a genome structure of colon cancer, so that the tissue origin of diseased cells 2 is colon cancer (i.e., colon).

In more detail, the cell origin obtainment unit 150 may determine similarity using the peak pattern of the state modification region of the genome structure by selecting one or more methods from three methods described below alone or in combination.

First, the cell origin obtainment unit 150 may obtain the tissue origin of the diseased cells by comparing a peak position of the genome structure stored in the genome structure state pattern DB for each tissue with a peak position of the genome structure of the collected cells.

FIG. 8 is a diagram for describing the comparison of genome structure patterns using peak positions of the genome structure according to the present invention.

That is, the cell origin obtainment unit 150 may expand the range to a gene regulatory region including tissue/disease-specific peaks, and determine that if peaks of diseased cells are present in the corresponding gene region and the gene regulatory region, the peaks match each other.

Referring to FIG. 8, since diseased cells 1 and diseased cells 2 are included in the gene region and the gene regulatory region including peaks of a stomach cancer-specific ABC gene, it is determined that the diseased cells match the peaks of the stomach cancer-specific ABC gene, and since diseased cells 3 are not included in the gene region and the gene regulatory region including the peaks of the stomach cancer-specific ABC gene, it is determined that the diseased cells mismatch the peaks of the stomach cancer-specific ABC gene.

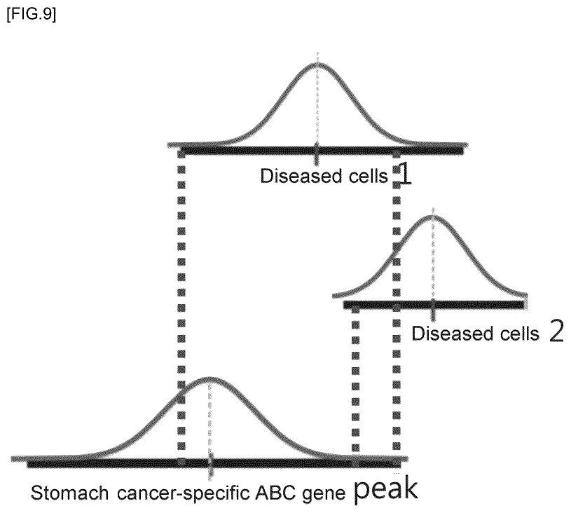

Second, the cell origin obtainment unit 150 may obtain the tissue origin of the diseased cells using the degree in which a peak region of the genome structure stored in the genome structure state pattern DB for each tissue overlaps with a peak region of the genome structure of the collected cells. For example, as illustrated in FIG. 9, the cell origin obtainment unit 150 may obtain the tissue origin of the diseased cells based on the ratio in which the peak regions of the genome structures overlap with each other.

FIG. 9 is a diagram for describing the comparison of genome structure patterns using a peak region overlap ratio of the genome structure according to an exemplary embodiment of the present invention.

In an exemplary embodiment, the cell origin obtainment unit 150 may determine that two peaks match each other when the length of a crossing area between samples is 50% or more of the peak region length of each sample, by using a “reciprocal >50% overlap” method used for comparison of general range regions.

Referring to FIG. 9, diseased cells 1 satisfy the reciprocal 50% overlap with the peak region of the stomach cancer-specific ABC gene, so that it is determined to match the stomach cancer-specific ABC gene peak, and diseased cells 2 do not satisfy the reciprocal 50% overlap, so that it is determined not to match the stomach cancer-specific ABC gene peak.

Third, the cell origin obtainment unit 150 may obtain the tissue origin of the diseased cells by comparing a matrix obtained based on a peak score of the genome structure stored in the genome structure state pattern DB for each tissue with a matrix obtained based on a peak score of the genome structure of the collected cells.

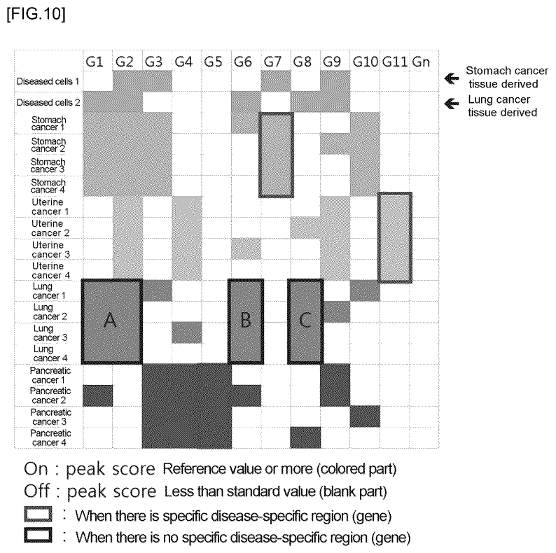

FIG. 10 is a diagram for describing the comparison of genome structure patterns using peak scores of the genome structure according to the present invention.

That is, the cell origin obtainment unit 150 sets a reference value of the peak score for all gene regions and then makes a matrix with Off if the peak score corresponding to the gene is lower than the reference value and On if it is higher than the reference value. In addition, the cell origin obtainment unit 150 may find a tissue/disease with a similar pattern to the diseased cells by comparing the On/Off values of the diseased cells based on the matrix.

Referring to FIG. 10, diseased cells 1 were determined to match stomach cancer tissue because a peak was found in G7, a stomach cancer-specific gene, and diseased cells 2 were determined to match lung cancer tissue based on a combination of genes (A, B, and C are all On) because there was no tissue/disease-specific genes.

The cell quantity obtainment unit 160 analyzes a state modification region of the genome structure based on the standard genome structure state pattern DB and the genome structure state pattern DB for each tissue stored in the storage unit 110, and obtains the quantity of the diseased cells.

That is, the cell quantity obtainment unit 160 may obtain the quantity of the diseased cells by calculating the number of diseased cells compared to the total number of cells, by using the number of sequences obtained for a state modification region of a specific genomic structure of the diseased cells and the number of sequences obtained for a state modification region of a specific genomic structure of the normal cells, based on the standard genome structure state pattern DB and the genome structure state pattern DB for each tissue.

In more detail, the cell quantity obtainment unit 160 compares the standard genome structure state pattern DB and the genome structure state pattern DB for each tissue to calculate the number Dr of sequences decoded for a state modification region of a genome structure specific to diseased cells that are not present in normal cells (white blood cells) using Equation 1 below.

$${D}_{r} = {\sum }_{k = 1}^{n} {D}_{k} \qquad [ Equation 1 ]$$

$$Normalized sequence number of disease cell - specific region 1 ( {D}_{1} ) = \frac{Number of sequences aligned in diseased cell - specific region 1 \times Sequence length \times {10}^{6}}{Total number of diseased cell - specific regions \times Length of diseased cell - specific region 1}$$

Here, n represents the total number of disease cell-specific regions.

The cell quantity obtainment unit 160 compares the standard genome structure state pattern DB and the genome structure state pattern DB for each tissue to calculate the number Cr of sequences decoded for a state modification region of a genome structure specific to normal cells that are not present in diseased cells using Equation 2 below.

$${C}_{r} = {\sum }_{k = 1}^{n} {C}_{k} \qquad [ Equation 2 ]$$

$$Normalized sequences number of normal cell - specific region 1 ( {C}_{1} ) = \frac{Number of sequences aligned in normal cell - specific region 1 \times Sequence length \times {10}^{6}}{Total number of normal cell - specific regions \times Length of normal cell - specific region 1}$$

Here, m represents the total number of normal cell-specific regions.

At this time, the profiling of a diseased cell specific region/normal cell specific region profile is performed through the following process:
First, sequences produced through ATAC-seq are aligned to the human reference genome.
After alignment, a filtering process of a generated binary alignment map (BAM) file is performed (BAM file processing).
A peak region of each sample is profiled using a Peak calling program.
A diseased cell-specific region/normal cell-specific region is found by comparing the standard genome structure state pattern DB and the genome structure state pattern DB for each tissue.
The number of sequences corresponding to the diseased cell-specific region/normal cell-specific region is calculated.

The cell quantity obtainment unit 160 may obtain the quantity of the diseased cells by calculating the number (concentration) of diseased cells compared to the total number of cells through the following Equation 4, based on the number Dr of sequences decoded for the state modification region of the genome structure specific to the diseased cells and the number Cr of sequences decoded for the state modification region of the genome structure specific to the normal cells.

In other words, the ratio of the number of diseased cells to the total number of cells may be expressed as Equation 3 below.

$$\frac{The number of disease cells}{The total number of cells} = \frac{Total diseased cell - specific normalized sequence number ( {D}_{r} )}{\begin{matrix}Total diseased cell - specific normalized sequence number ( {D}_{r} ) + \\ Total normal cell - specific normalized sequence number ( {C}_{r} )\end{matrix}} \qquad [ Equation 3 ]$$

Therefore, the number of diseased cells may be calculated using Equation 4 below.

$$The number of diseased cells = \frac{Total disased cell - specific normalized sequence number ( {D}_{r} )}{\begin{matrix}Total diseased cell - specific normalized sequence number ( {D}_{r} ) + \\ Total normal cell - specific normalized sequence number ( {C}_{r} )\end{matrix}} \times The total number of cells \qquad [ Equation 4 ]$$

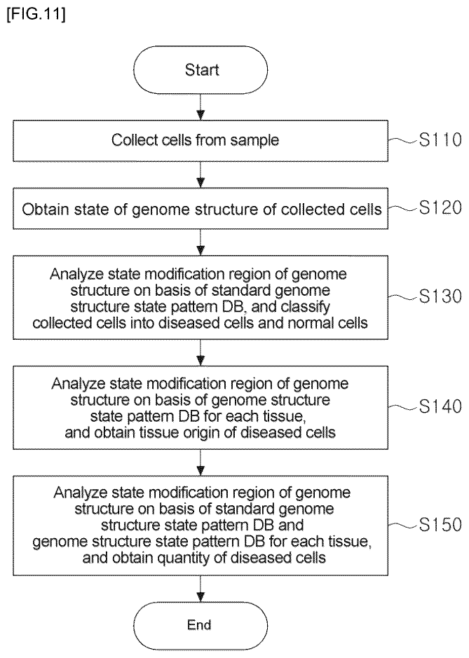

Then, a method for analyzing cells by using state modification information of a chromosome structure according to a preferred embodiment of the present invention will be described with reference to FIG. 11.

FIG. 11 is a flowchart for describing a method for analyzing cells by using state modification information of a chromosome structure according to a preferred embodiment of the present invention.

Referring to FIG. 11, the cell analysis apparatus 100 collects the cells from the sample of the subject (S110).

In addition, the cell analysis apparatus 100 obtains the state of the genome structure of the collected cells (S120). That is, the cell analysis apparatus 100 may confirm the sequence patterns, structures, and the like on the genome through genome decoding or genotyping of the collected cells.

Thereafter, the cell analysis apparatus 100 analyzes a state modification region of the genome structure based on the standard genome structure state pattern DB, and classifies the collected cells into diseased cells and normal cells (S130). That is, the cell analysis apparatus 100 may classify the collected cells into diseased cells and normal cells by comparing the genome structure stored in the standard genome structure state pattern DB with the genome structure of the collected cells, based on the number of sequences and peaks of the state modification region of the genome structure.

In addition, the cell analysis apparatus 100 analyzes a state modification region of the genome structure based on the genome structure state pattern DB for each tissue, and obtains the tissue origin of the diseased cells (S140). That is, the cell analysis apparatus 100 may obtain the tissue origin of the diseased cells by comparing the genome structure stored in the genome structure state pattern DB for each tissue with the genome structure of the collected cells, based on a peak pattern of the state modification region of the genome structure.

In more detail, the cell analysis apparatus 100 may determine similarity using the peak pattern of the state modification region of the genome structure by selecting one method from three methods described below.

First, the cell analysis apparatus 100 may obtain the tissue origin of the diseased cells by comparing a peak position of the genome structure stored in the genome structure state pattern DB for each tissue with a peak position of the genome structure of the collected cells.

Second, the cell analysis apparatus 100 may obtain the tissue origin of the diseased cells, based on a ratio in which a peak region of the genome structure stored in the genome structure state pattern DB for each tissue overlaps with a peak region of the genome structure of the collected cells.

Third, the cell analysis apparatus 100 may obtain the tissue origin of the diseased cells by comparing a matrix obtained based on a peak score of the genome structure stored in the genome structure state pattern DB for each tissue with a matrix obtained based on a peak score of the genome structure of the collected cells.

Thereafter, the cell analysis apparatus 100 analyzes a state modification region of the genome structure based on the standard genome structure state pattern DB and the genome structure state pattern DB for each tissue and obtains the quantity of the diseased cells (S150). That is, the cell analysis apparatus 100 may obtain the quantity of the diseased cells by calculating the number of diseased cells compared to the total number of cells, by using the number of sequences obtained for a state modification region of a specific genomic structure of the diseased cells and the number of sequences obtained for a state modification region of a specific genomic structure of the normal cells, based on the standard genome structure state pattern DB and the genome structure state pattern DB for each tissue.

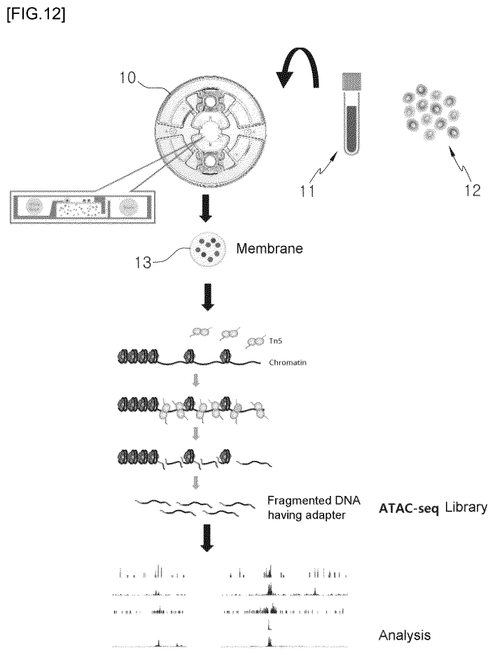

Hereinafter, even when the amount of diseased cells is small by analyzing the state modification region of the genome structure of the collected cells, it will be described again through experimental data that it is easy to determine whether the collected cells are diseased cells or normal cells. FIG. 12 is a flowchart illustrating steps of an experiment performed to obtain experimental data.

In the experiment, the diseased cells were isolated from an experimental group sample using a device 10 capable of isolating cancer cells such as CTC from the blood (for example, CD-CTC Duo Disc™ from Clinomics). As a control group, peripheral blood mononuclear cells (PBMC) obtained from the whole blood of an ordinary person was prepared as a normal cell sample (negative control sample). In addition, a cancer cell line was prepared as a diseased cell sample (positive control sample). Ovarian cancer_SK-OV-3 was used as the cancer cell line, and an experimental group sample was prepared by spiking the cancer cell line in PBMC obtained from the whole blood of the ordinary person for each number. The number of cancer cell lines spiked for each sample was shown in Table 1.

TABLE 1

| Experimental group sample | The number of cancer cell lines |
|---|---|
| Sample 1 | 1 |
| Sample 2 | 10 |
| Sample 3 | 100 |

First, a normal cell sample 11, a diseased cell sample 12, and experimental group samples 1 to 3 were added to the device 10 to separate the cells for each sample, respectively. In addition, a membrane 13 containing the cells separated from each sample was taken out of the device 10 and lysed, and then an ATAC-seq library was constructed, and the sequencing data was analyzed to select a different region between the samples.

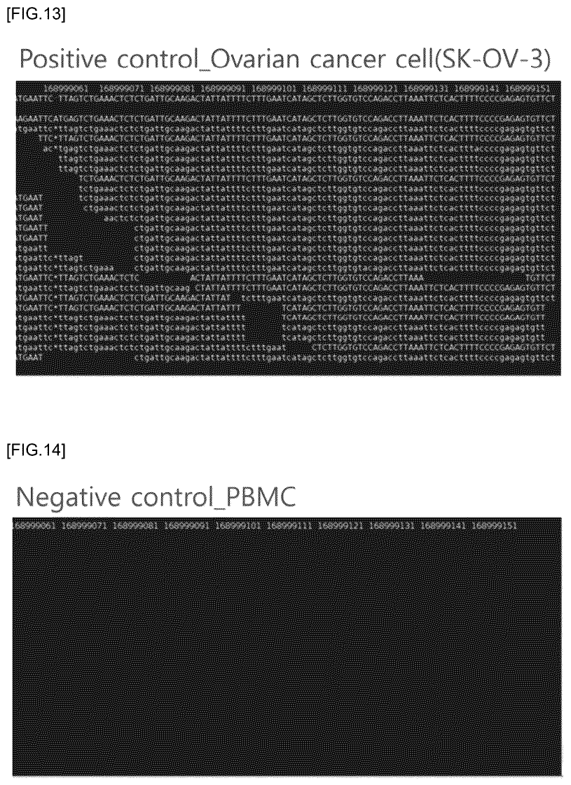

FIG. 13 illustrates a result of analyzing sequencing data for cells isolated from the diseased cell sample 12, and FIG. 14 illustrates a result of analyzing sequencing data for cells isolated from the normal cell sample 11. By selecting sequences that were detected in the data of FIG. 13, but not in the data of FIG. 14, a different region between the diseased cell sample and the normal cell sample may be determined.

That is, it may be seen that a region containing sequences which were detected in the data of FIG. 13, but not in the data of FIG. 14 is a genomic region in which transcriptional expression did not occur in the normal cell sample, and a euchromatin region in which transcriptional expression occurred in the diseased cell sample.

As such, by comparing FIGS. 13 and 14, by selecting sequences that are not detected in the normal cell sample, but detected in the diseased cell sample, the state modification region of the genome structure may be selected and peaks (the peaks described above with reference to FIG. 5) may be found in the corresponding region of the diseased cells. In addition, Samples 1 to 3 may be classified into diseased cells and normal cells by analyzing whether the corresponding peaks were detected in Samples 1 to 3.

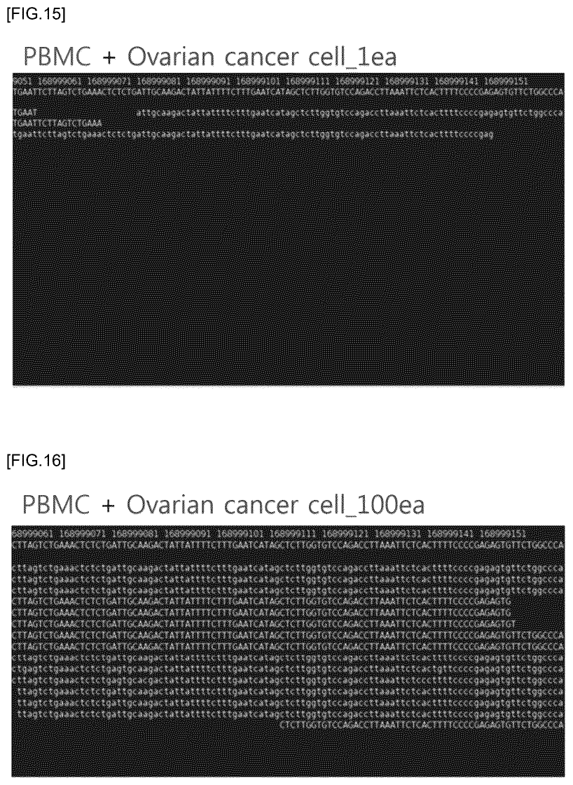

FIG. 15 illustrates a result of analyzing sequencing data for cells isolated from Sample 1, and FIG. 16 illustrates a result of analyzing sequencing data for cells isolated from Sample 3. Referring to FIG. 15, it may be seen that the euchromatin region detected only in the sequencing data of the diseased cell sample 12 compared to the normal cell sample 11 is detected well even in Sample 1 in which ovarian cancer_SK-OV-3 (1 ea) was spiked in PBMC. Therefore, according to the present invention, even when a small amount of diseased cells are actually present in the collected cells, the collected cells may be clearly classified into diseased cells through analysis of the state modification region of the genome structure.

In addition, as may be seen by comparing FIGS. 15 and 16, it may be seen that in Sample 3, in which ovarian cancer_SK-OV-3 (100 ea) was spiked into PBMC, the number (depth) of sequences in the corresponding euchromatin region was higher than that in Sample 1.

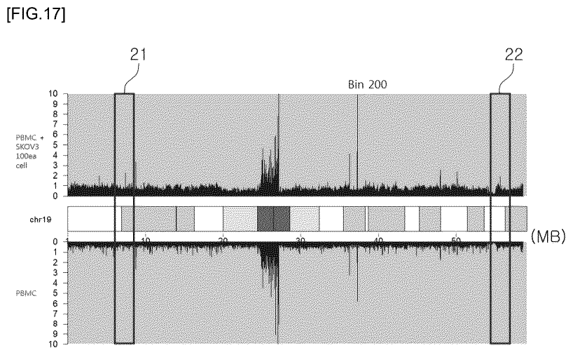

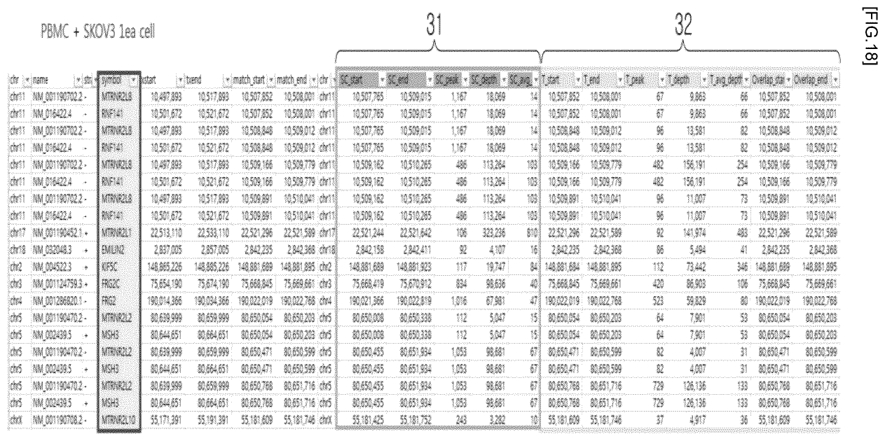

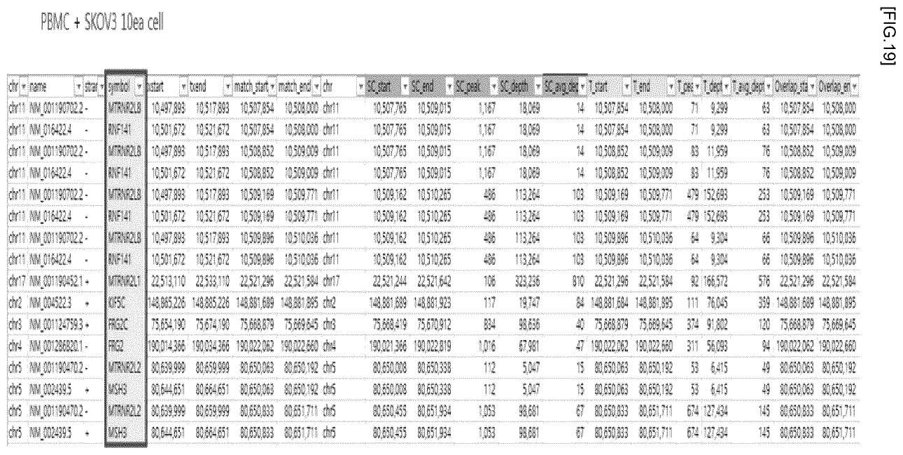

FIG. 17 is a graph showing comparing the sizes of peaks detected in sequencing data (illustrated in the lower part of the graph) of cells isolated from the normal cell sample 11 and sequencing data (illustrated in the upper part of the graph) of cells isolated from Sample 3 at positions of the same chromosome. In the graph of FIG. 17, an x-axis represents a chromosomal location of chromatin 19 (chr19) (unit: megabase, MB), and a y-axis represents a peak size in units of 200 base sequences (Bin 200).

When comparing the normal cell sample 11 and Samples 1 to 3, peaks shown only in the experimental group samples were identified to determine the corresponding sample as diseased cells. For example, in FIG. 17, peaks were detected for Sample 3 in a region 21 corresponding to 7 to 8 MB, but no peak was detected for the normal cell sample 11, and the peaks were detected only in Sample 3 in a region 22 corresponding to 55 to 56 MB, and thus Sample 3 may be determined as diseased cells.

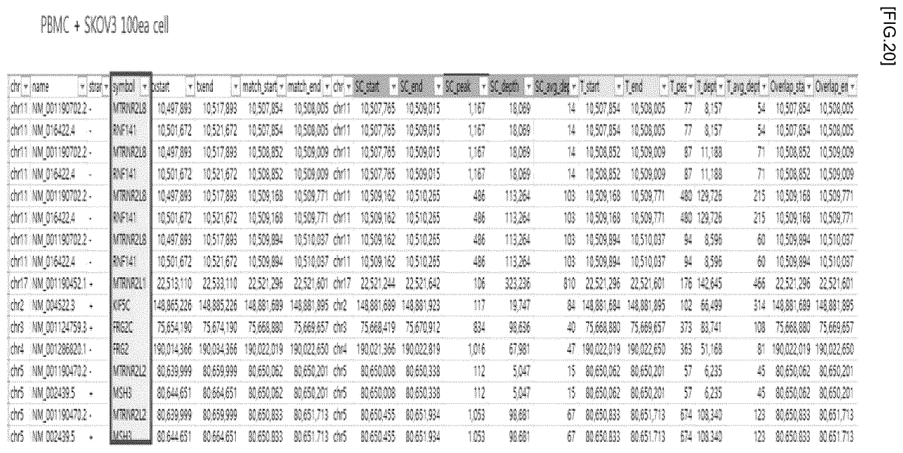

In this way, meaningful results may be obtained by statistical processing of the sequence of each region. FIGS. 18 to 20 are result data of analyzing sequences obtained by sequencing Samples 1 to 3 by the aforementioned method using a computer program (e.g., GEN RICH™), respectively. FIGS. 18 to 20 illustrate result data for Samples 1 to 3, respectively. In data tables of FIGS. 18 and 19, the first column represents the chromatin number, and the fourth column (indicated by a red box) represents the gene symbol.

When comparing the data tables of FIGS. 18 to 20, it may be seen that gene symbols in regions different from in the normal cell sample 11 are detected in a similar pattern in each sample. Referring to FIG. 18, data for each sample may be classified into 1) data 31 on sequence regions to be detected only in the diseased cell sample 12 compared to the normal cell sample 11, and 2) data 32 of sequences to be detected differently according to the amount of diseased cells even in sequences to be detected only in the diseased cell sample 12 compared to the normal cell sample 11.

That is, among the data 31 indicated in a green box in FIG. 18, values of the peak data in the third column and the sequence number (depth) data in the fourth column are shown equally even in the data tables of FIGS. 19 and 20. In addition, it may be seen that among the data 32 indicated in a yellow box in FIG. 18, values of the peak data in the third column and the sequence number (depth) data in the fourth column are gradually larger in FIGS. 19 and 20 in which the content of diseased cells is gradually increased. Through the data of FIGS. 18 to 20, the diseased cell sample may be distinguished from the normal cell sample, and the number of sequences and reads in the distinguished sequence regions may be confirmed.

The present invention may be implemented as a computer readable code in a computer readable recording medium. The computer readable recording medium includes all kinds of recording devices for storing data which may be read by a computer. Examples of the computer readable recording medium include a ROM, a RAM, a CD-ROM, a magnetic tape, a floppy disk, an optical data storage device, and the like.

While the preferred exemplary embodiments of the present invention have been illustrated and described above, the present invention is not limited to the aforementioned specific preferred exemplary embodiments, various modifications may be made by a person with ordinary skill in the technical field to which the present invention pertains without departing from the subject matters of the present invention that are claimed in the claims, and these modifications are included in the scope of the claims.

1. A method for analyzing cells by using state information of a chromosome structure, the method comprising:
obtaining a state of a genome structure of cells collected from a sample;
analyzing a state modification region of the genome structure based on a pre-stored standard genome structure state pattern DB, and classifying the collected cells into diseased cells and normal cells;
analyzing a state modification region of the genome structure based on a pre-stored genome structure state pattern DB for each tissue, and obtaining the tissue origin of the diseased cells; and
analyzing a state modification region of the genome structure based on the standard genome structure state pattern DB and the genome structure state pattern DB for each tissue, and obtaining the quantity of the diseased cells.

2. The method of claim 1, wherein the classifying consists of classifying the collected cells into the diseased cells and the normal cells by comparing the state of the genome structure stored in the standard genome structure state pattern DB with the state of the genome structure of the collected cells, based on the number of sequences and peaks of the state modification region of the genome structure.

3. The method of claim 1, wherein the obtaining of the tissue origin consists of obtaining the tissue origin of the diseased cells by comparing the state of the genome structure stored in the genome structure state pattern DB for each tissue with the state of the genome structure of the collected cells, based on a peak pattern of the state modification region of the genome structure.

4. The method of claim 3, wherein the obtaining of the tissue origin consists of obtaining the tissue origin of the diseased cells by comparing a peak position of the genome structure stored in the genome structure state pattern DB for each tissue with a peak position of the genome structure of the collected cells.

5. The method of claim 3, wherein the obtaining of the tissue origin consists of obtaining the tissue origin of the diseased cells based on a ratio in which a peak region of the genome structure stored in the genome structure state pattern DB for each tissue overlaps with a peak region of the genome structure of the collected cells.

6. The method of claim 3, wherein the obtaining of the tissue origin consists of obtaining the tissue origin of the diseased cells by comparing a matrix obtained based on a peak score of the genome structure stored in the genome structure state pattern DB for each tissue with a matrix obtained based on a peak score of the genome structure of the collected cells.

7. The method of claim 1, wherein the obtaining of the quantity of the diseased cells consists of obtaining the quantity of the diseased cells by calculating the number of diseased cells compared to the total number of cells, by using the number of sequences obtained for a state modification region of a specific genomic structure of the diseased cells and the number of sequences obtained for a state modification region of a specific genomic structure of the normal cells, based on the standard genome structure state pattern DB and the genome structure state pattern DB for each tissue.

8. An apparatus for analyzing cells by using state information of a chromosome structure, the apparatus comprising:
a cell analysis unit configured to obtain a state of a genome structure of cells collected from a sample;
a cell classification unit configured to analyze a state modification region of the genome structure based on a pre-stored standard genome structure state pattern DB, and classify the collected cells into diseased cells and normal cells;
a cell origin obtainment unit configured to analyze a state modification region of the genome structure based on a pre-stored genome structure state pattern DB for each tissue, and obtain the tissue origin of the diseased cells; and
a cell quantity obtainment unit configured to analyze a state modification region of the genome structure based on the standard genome structure state pattern DB and the genome structure state pattern DB for each tissue, and obtain the quantity of the diseased cells.

9. The apparatus of claim 8, wherein the cell classification unit classifies the collected cells into the diseased cells and the normal cells by comparing the state of the genome structure stored in the standard genome structure state pattern DB with the state of the genome structure of the collected cells, based on the number of sequences and peaks of the state modification region of the genome structure.

10. The apparatus of claim 8, wherein the cell origin obtainment unit obtains the tissue origin of the diseased cells by comparing the genome structure stored in the genome structure state pattern DB for each tissue with the genome structure of the collected cells, based on a peak pattern of the state modification region of the genome structure.

11. The apparatus of claim 10, wherein the cell origin obtainment unit obtains the tissue origin of the diseased cells by comparing a peak position of the genome structure stored in the genome structure state pattern DB for each tissue with a peak position of the genome structure of the collected cells.

12. The apparatus of claim 10, wherein the cell origin obtainment unit obtains the tissue origin of the diseased cells based on a ratio in which a peak region of the genome structure stored in the genome structure state pattern DB for each tissue overlaps with a peak region of the genome structure of the collected cells.

13. The apparatus of claim 10, wherein the cell origin obtainment unit obtains the tissue origin of the diseased cells by comparing a matrix obtained based on a peak score of the genome structure stored in the genome structure state pattern DB for each tissue with a matrix obtained based on a peak score of the genome structure of the collected cells.

14. The apparatus of claim 8, wherein the cell quantity obtainment unit obtains the quantity of the diseased cells by calculating the number of diseased cells compared to the total number of cells, by using the number of sequences obtained for a state modification region of a specific genomic structure of the diseased cells and the number of sequences obtained for a state modification region of a specific genomic structure of the normal cells, based on the standard genome structure state pattern DB and the genome structure state pattern DB for each tissue.
